(19) United States
(12) Patent Application Publication
(10) Pub. No.: US 2016/0005322 A1
(43) Pub. Date: Jan. 7, 2016

(54) Method And Computer Algorithm to Determine A User's Mental Agility, Memory and Emotional State Based on Recreational Media Content And Game Play

(71) Applicant: Bernard Anthony Xavier, Del Mar, CA (US)

(72) Inventor: Bernard Anthony Xavier, Del Mar, CA (US)

(21) Appl. No.: 14/322,120

(22) Filed: Jul. 2, 2014

(51) Int. Cl. G09B 7/00; A61B 5/16

(52) U.S. Cl. CPC G09B 7/00; A61B 5/165

(57) ABSTRACT

A computerized system to determine and predict the mental agility and mood of a user by compiling a list of questions based on recently viewed and or listened to recreational media content. Comparing the user's responses with their peer group and the user's historical record enables the system to determine a deviation in mental agility and or emotional state. Deviation from the expected response indicates a change in the user's mood or mental state. Alerts may be sent when these deviations are detected.

### RELATED APPLICATIONS

This application claims the priority of US provisional applications Nos. 61/842462, 61/842502, 61/842506, 61/842509, 61/842455, and 61/842481, all filed on Jul. 3, 2013. Each of these applications is herein incorporated by reference in their entirety for all purposes.

### BACKGROUND OF THE INVENTION

1. Field of the Invention

The present invention is in the field of medical care, more particularly the present invention is in the area of computerized monitoring of a user's emotional state, mental health, and physical health.

2. Description of Related Art

Present systems to determine mental health rely on a user answering a set of predetermined questions based on images and statements. These images and statements are selected by historical studies and research carried out by psychologists and psychiatrists, or other professionals in the field. The answers are recorded and statistics are produced. The statistics are then used to determine and predict the user's emotional state, mental health, and physical health.

### SUMMARY OF THE INVENTION

In accordance with the present invention a method to test a user's mental agility, memory, and emotional state based on media content. The system presents media content to the user on a display device. The system derives a list of questions for the user based on the presented media content. For example if the user has recently watched a documentary about African Lions the system could ask where the program took place, how many animals were present, etc. These questions can be repeated at varying frequency over the coming week. The questions are displayed to the user on the display device. The user selects answers on a separate input device. The display device and input device are controlled by a remote server computer. The remote server computer records the user's response time, answers and accuracy of the answers. The remote server computer uses the recorded data to calculate parameters representing the user's mental agility and memory. If the result of the calculation shows a difference to a set of predefined deviation parameters, the system signals that an action needs to be taken. The underlying principle is that the user should have the same or similar emotional reaction to similar content. In addition, the system may ask questions related to the user's emotional state. The answers to said questions are indicative of the user's mood.

### BRIEF DESCRIPTION OF THE DRAWINGS

FIG. 1 shows a computerized system for measuring a patient's responses and viewing recreational media.

FIG. 2 shows a flowchart describing the process of asking questions based on media content for the purpose of determining the user's mental agility, memory, and mood.

FIG. 3 shows a server controlled remote entry system with separate input and output devices.

FIG. 4 shows a server controlled remote system for a single user.

FIG. 5 shows areas of consideration for user choices to predict the user's emotional state.

FIG. 6 shows a flowchart describing how to process and compare the user's choice or response to a peer group or the user's historical choices and responses.

FIG. 7 shows a method for implementing a system for employing statistical techniques to estimate the statistics of time varying user input data and during game play.

### DETAILED DESCRIPTION OF THE INVENTION

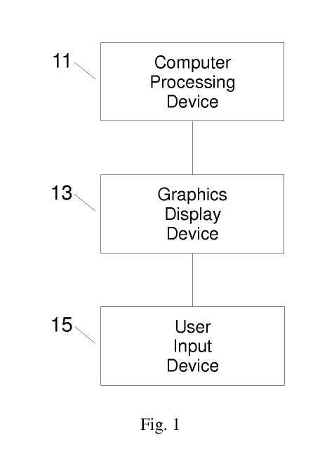

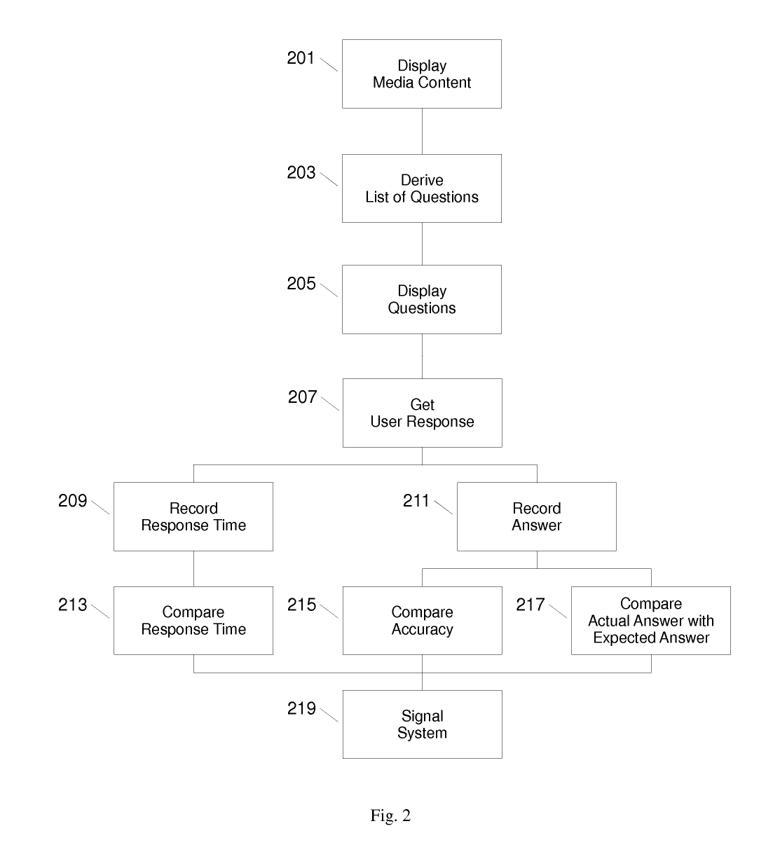

Referring now to the invention in more detail, a typical setup of the present invention is illustrated in FIG. 1 and FIG. 2.

FIG. 1 shows a computerized system for measuring a patient's responses and viewing recreational media, the system comprising a computer processing device 11, a graphics display device 13 and user input device 15. The computer program executed at said computer processing device generates output data to be displayed on a graphical display device and processes input from an input device. The output data comprises recreational media content and a list of questions about said recreational media content. User input device 15 is adapted to receive user's input, including the responses to said questions shown on said graphic display device, and to forward such responses to said computer processing device. The responses are processed by a computer program run on said computer processing device and the parameters representing the mental, agility, or emotional state of the user is determined or estimated.

FIG. 2 describes one embodiment of the process of asking questions based on media content for the purpose of determining the user's mental agility, memory, and mood. The method comprises the steps of:

201 to display media content in the form of graphical or audio content on the graphical display device 13;

203 to derive a list of questions based on the displayed media content. Said questions are related to the storyline, the characters, and the locations of the media content;

205 to display said questions derived in process 203 on the graphical display device 13;

207 to get the user's response to the questions displayed in process 205;

209 to record the response time between the display of the media questions in process 205 and the user responses in process 207;

211 to record the answer given in process 207;

213 to compare the response time from process 209 to response times of similar questions, and similar users for the same questions;

215 to compare the accuracy of the response from process 211 to the accuracy of responses from similar questions, and similar users for the same questions;

217 to compare the answer given by the user to the expected answer based on the user's background and history of answering similar questions.

219 to signal any deviation of the measured parameters from processes 213, 215 and 217 to the system.

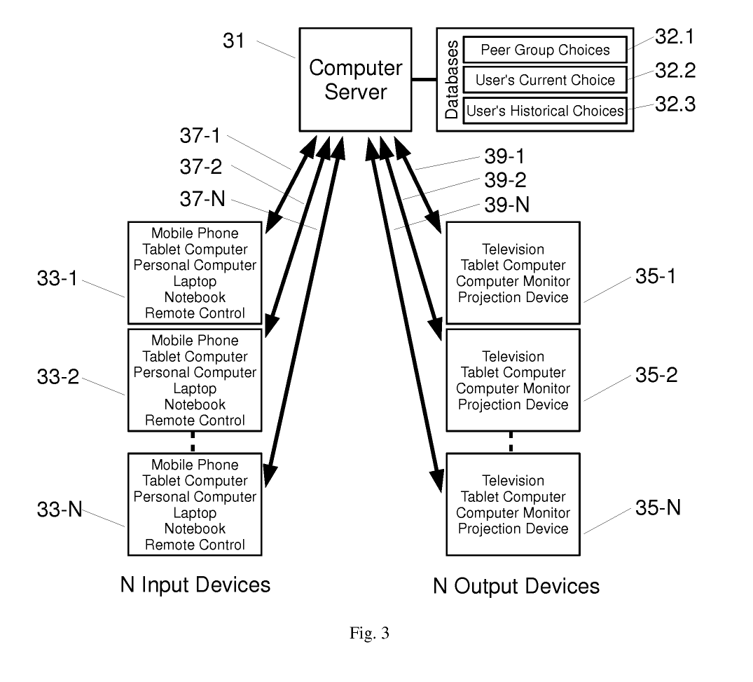

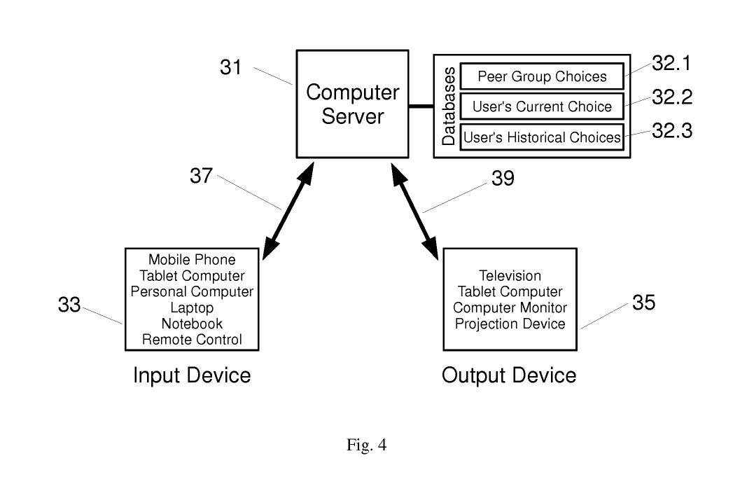

Referring now to the invention in more detail, a typical setup of the present invention is illustrated in FIG. 3 to FIG. 4.

In FIG. 3 there is shown the computer server 31, a set of N input devices 33-1, 33-2 up to 33-N, and a set of N output devices 35-1, 35-2, up to 35-N. The N input devices are connected to the computer server 31 via communication links 37-1, 37-2, up to 37-N. The N output devices are connected to the computer server 31 via communications links 39-1, 39-2, up to 39-N. The user's current choice is stored in a database 32.2 “User's Current Choice”. The user's previous choices are stored in a database 32.1 “User's Historical Choices”. The choices of people in the user's peer group are stored in a database 32.3 “Peer Group Choices”.

In FIG. 4 there is shown a communication setup for a single user. The user makes entertainment choices by selecting an option displayed on input device 33. The data are transmitted to the computer server 31 via communication link 37. The computer server 31 stores the user's selection. The computer server 31 transmits entertainment data via communication link 39 to the output device 35. Output device 35 displays the entertainment data to the user.

A communication link 37 that transports data from the user's input device 33 to the server 31 shall be called “uplink”. A communication link 39 that transports data from the server 31 to the user's output device 35 shall be called “downlink”.

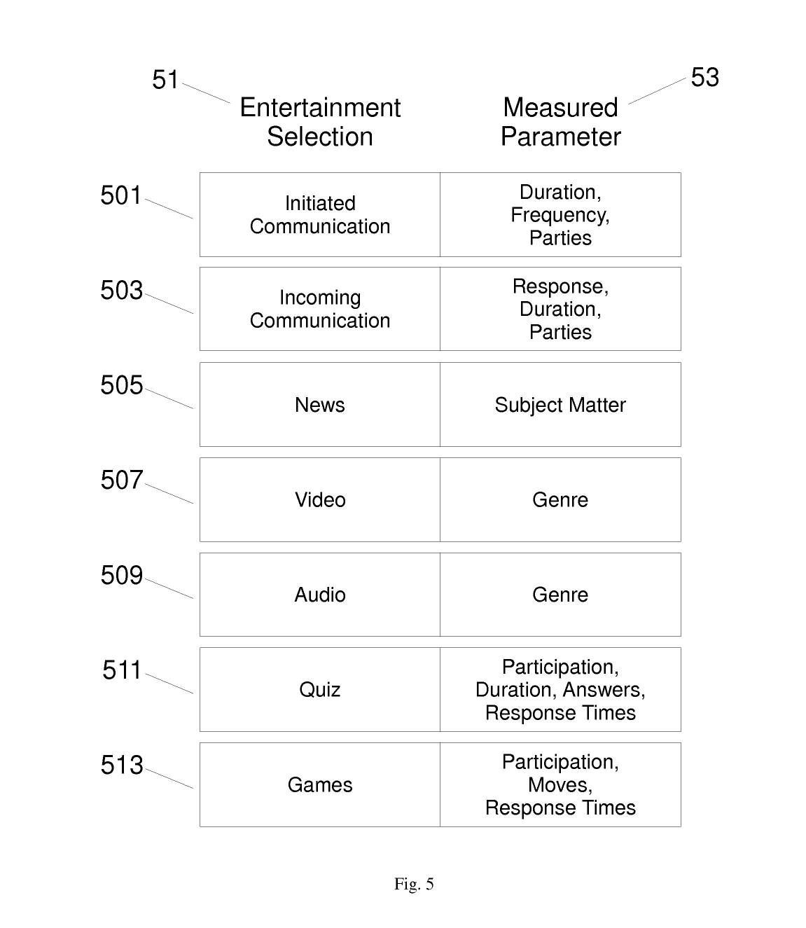

FIG. 5 shows areas of consideration for user choices to predict the user's emotional state. In FIG. 5 there is shown a list of entertainment selections 51. One or more measured parameters 53 is specified for each entertainment selection 51, comprising, for example the following selections:

Still referring to FIG. 5, there is shown a selection “Initiated Communication” 501. Initiated communication 501 means the user initiates the communication by making a selection on the input device 33. Initiated communications 501 include any combination of communications via voice, text, and video. The computer server 31 stores the duration of the communication, the frequency of the selection, and the parties participating in the communication.

Still referring to FIG. 5, there is shown a selection “Incoming Communication” 503. Incoming communication 503 means the user receives a communication. Incoming communications 503 include any combination of communications via voice, text, and video. The user chooses to accept or reject the communication by selection a response on the input device 33. The computer server 31 stores the response made by the user. The computer server 31 also stores the duration of the communication, and the parties participating in the communication.

Still referring to FIG. 5 there is shown a selection “News”505. News 505 include spoken and written content via any combination of voice, text, and video. The user chooses a news item by making a selection on the input device 33. The computer server 31 stores the subject matter of the selection made by the user.

Still referring to FIG. 5 there is shown a selection “Videos”507. Videos 507 include television programming, movies, shows, and other audiovisual content. The user chooses a video by making a selection on the input device 33. The computer server 31 stores the genre of the selection made by the user.

Still referring to FIG. 5 there is shown a selection “Audio”509. Audio 509 includes verbal, vocal, or orchestral audio content such as songs, concerts, audio books, and discussions. The user chooses the audio content by making a selection on the input device 33. The computer server 31 stores the genre of the selection made by the user.

Still referring to FIG. 5 there is shown a selection “Quiz”511. Quiz 511 includes mind challenges such as questions about mathematical calculations, trivia, or items of personal interest. The user chooses to participate in a quiz by making a selection on the input device 33. The computer server 31 stores the number of times the user enters the selection “Quiz” 511 as a measure of participation. The computer server 31 also stores the duration of the user's participation at a quiz. For each question, the computer server 31 also stores the user's answer, and response time.

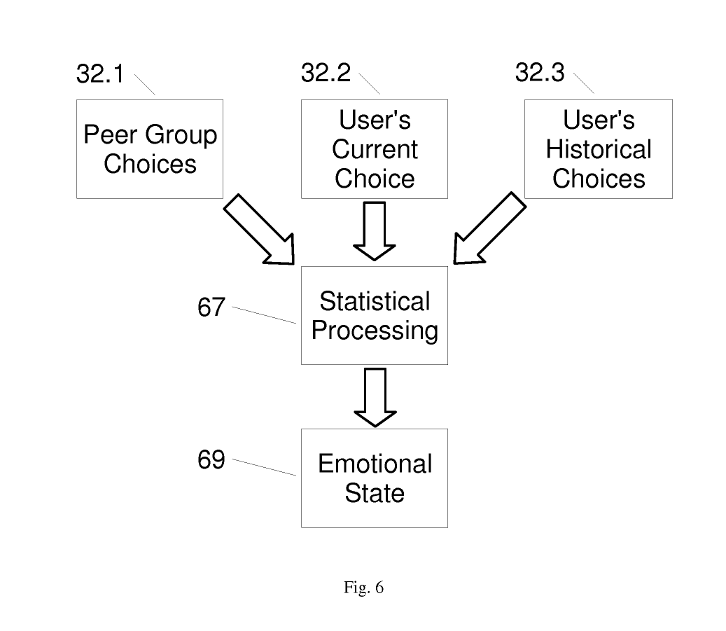

Still referring to FIG. 5 there is shown a selection “Games”513. Games 513 include single player and multi-player games such as Bingo, Black Jack, or Poker. The computer server 31 stores the number of times the user enters the selection “Games” 511 as a measure of participation. The computer server 31 also stores the duration of the user's participation at a game. The computer server 31 also stores the user's moves, and response times. FIG. 6 shows a flowchart describing how to process and compare the user's choice or response to a peer group or the user's historical choices and responses. A “Peer Group Choice” database 32.1 comprises the data of the entertainment choices made by a group of users with similar characteristics as the user. “User's Choice” database 32.2 comprises the data of the current entertainment choices made by the user. “User's Historical Choice” database 32.3 that is the record of the historical entertainment choices made by the user.

In step 67 “Statistical Processing” a statistical analysis of the peer group choice 32.1, user's current choice 32.2, and user's historical choice 32.3 is carried out. In step 69, the user's emotional state based on the output of the statistical processing 67 is determined or estimated, using appropriate algorithms.

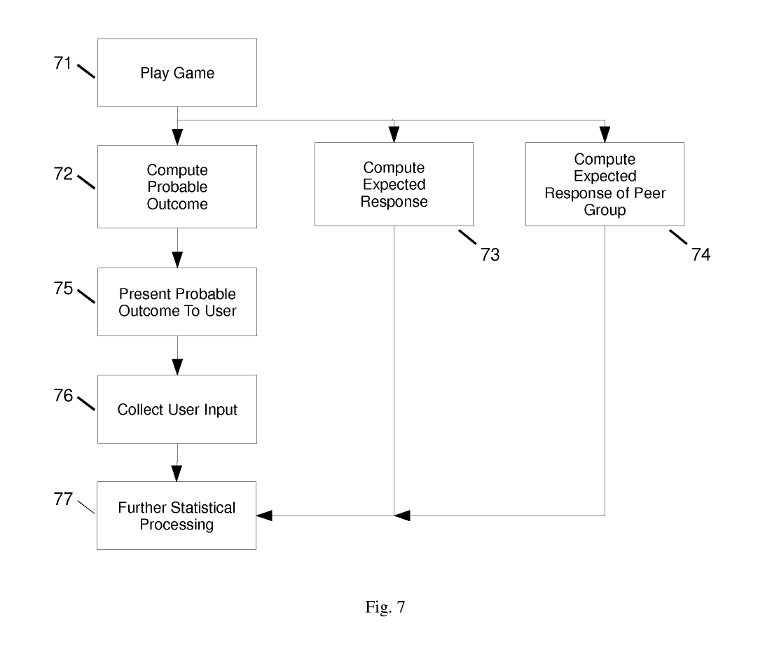

FIG. 7 shows a method according one embodiment of the invention employing statistical analysis techniques to estimate the statistics of time varying user input data and during game play.

Process 71 provides a user with a means to play an interactive computer game. Process 72 computes the expected outcome and response of the user. Said outcome and response is based on the previous states of the game and the prevailing conditions the user is experiencing. Process 73 computes the expected outcome of the game based on the user's past responses to similar game conditions and game states. The process 73 to compute the expected response is optional.

Process 74 computes the expected outcome or user's response to the prevailing conditions and game states based on historical data of users who have similar demographics as the user and who experienced similar conditions as the user is currently experiencing. The process 74 to compute the expected outcome based on peer data is optional.

Process 75 predicts the outcome of the game. The process 75 may advise the user of the next action to increase the probability for the user to win the game. The predicted outcome may be displayed on a screen or played through an audio system to the user.

Process 76 collect the user input data. The user may enter data via a touch screen device, a mouse or pointing device, a pen, a keyboard or via speaking to a device equipped with speech recognition capabilities. The user input data are stored in a memory device. The nature of the input and the time and or the time separation between successive samples is stored.

Process 77 performs additional statistical processing and determines the events and actions the computer system performs next.

Operation—FIG. 1 and FIG. 2 and FIG. 3

A computer processing device 11 displays media content to a user on a graphics display device 13. The user interacts with the computer processing device 11 using a user input device 15. The user input device 15 can be a remote control, a tablet PC, a mouse, a pointing device, a keyboard, or any other type of user input device.

The computer processing device 11 displays the media content to the user 201. The computer processing device 11 derives and or receives a list of questions 203 based on the media content. After the user has watched the media content, the computer processing device 11 displays the questions 205 and possible answers on the graphics display device 13. The user selects an answer to the question on the user input device 15. The computer processing device 11 receives the user's responses 207 from the user input device 15.

The computer processing device 11 records the response time 209. The response time is the time elapsed between the presentation of the answers to the user and the user selecting an answer. The computer processing device 11 records the user's answer 211.

The computer processing device 11 comprises a recorded history of response times to the same question by similar users. The computer processing device 11 contains a recorded history of response times to similar or the same questions by the user. The computer processing device compares the history of response times to the current response time 213.

The computer processing device 11 comprises a recorded history of the accuracy of answers to the same question by similar users. The computer processing device 11 comprises a recorded history of the accuracy of answers to similar or the same questions by the user. The accuracy rating is defined by how close the answer is to the correct answer. E.g. the correct answer receives an accuracy rating of 1. An answer that is incorrect, but close to the correct answer, receives a lower accuracy rating, e.g. 0.8. A response that is the opposite of the correct response receives a very low accuracy rating, e.g. 0. The computer processing device 11 compares the history of accuracy ratings to the current accuracy rating 215.

The computer processing device 11 compares the actual answer with the expected answer 217.

The computer processing device 11 uses the comparison data calculated and stored above in steps 213, 215, and 217 to determine deviations from previous calculations. The computer processing device uses predetermined thresholds of deviations from the average and flags significant deviations. A flag initiates a signaling procedure to alert the user or any other involved party of the deviation 219.

Operation—FIG. 3 to 6

The user makes entertainment choices on the input device 33. Input device 33 uses uplink 37 to send the user's choices to the server 31. Upon reception of the uplink data, the server 31 appends the current content of “User's Current Choice” to database 12.3 “User's Historical Choices”. The server then replaces the content of “User's Current Choice” with the received data. The server 31 uses the downlink 39 to send output data to the user's output device 35.

Other members in the same peer group as the user make entertainment choices on their input devices 33. The server appends these entertainment choices to the database 32.1 “Peer Group Choices”.

The statistical processing 67 compares data from database “User's Historical Choices” 32.3 and database “Peer Group Choices” 32.1 to the data of database “User's Current Choice” 32.2. The statistical processing 67 uses a computer algorithm to calculate the user's emotional state 69.

Operation—FIG. 7

The user plays a game presented by process 71. The user's responses to the current game condition and game state are recorded. The user's past responses to similar game conditions and game states are recorded as well. The expected response of the user is calculated using past user responses (step 72). Optionally, the expected outcome of the game is calculated (step 73). The expected response of the user is also calculated separately by using the historical responses of a peer group of users with similar demographics (step 74). The outcome of the game is predicted. The prediction can be used to guide the user in the game by suggesting the next action (step 75). The user may enter game moves via an input device such as a touch screen device, a mouse or pointing device, a pen, a keyboard or via speaking to a device equipped with speech recognition capabilities (step 76). Memory devices are used to store present and past user inputs. Statistical processing is performed to determine the mental state, or mood of the user (step 77).

Although the description above contains many specifications, these should not be construed as limiting the scope of the invention but as merely providing illustrations of some of the presently preferred implementations of this invention. For example, different algorithms may be used for calculating the user's mental agility, memory, and mood. For example, the input devices can be of different nature, such as touch-less sensors; the computer server can be split into several devices, additional entertainment selections can be used, etc. For example, different algorithms may be used for calculating the user's mental state.

While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. The invention should therefore not be limited by the above described embodiment, method, and examples, but by all embodiments and methods within the scope and spirit of the invention as claimed.

Thus the scope of the invention should be determined by the appended claims and their legal equivalents, rather than by the examples given.

### REFERENCE NUMERALS IN DRAWINGS

10113 Server or host computer

103—Gateway to wide area network

105—User input device

107—User output device

10913 Communication link between gateway and user input device

111—Communication link between gateway and user output device

113—Communication link between user input device and user output device

21—Uplink between user input device and computer server

23—Downlink between computer server and user input device

25—Downlink between computer server and user output device

31—Generation of a set of unique identification numbers

33—Assignment of an identification number to an input device

35—Assignment of an identification number to an output device

1. A method of determining mental state of a person in a computer system comprising a computer processing device, a user input device and a user output device, the method comprising:
said computer processing device providing content to said user output device;
said computer processing device providing a set of questions related to said content on said user output device;
said user input device receiving user input in response to questions of said set of questions, and transferring said responses to said computer processing device:
said computer processing device recording said user responses and measured parameters;
comparing said measured parameters and said responses with expected measured parameters and expected response, and
outputting data representing the mental stage of the user.

2. A method as in claim 1, wherein said comparing comprises comparing the user's current response with users historical responses.

3. A method as in claim 2, wherein said comparing comprises comparing the user's current response with a peer group responses.

4. A method as in claim 3, wherein said content is a computer game.

5. A method as in claim 4, said measured parameters include a response time.

6. A method as in claim 3, wherein said content is entertainment media content.

7. A method as in claim 6, wherein said content is a quiz.

8. A method as in claim 7, wherein said measured parameters include response time and accuracy.

9. A method as in claim 5, wherein said content is news.

10. A method as in claim 6, wherein said measured parameters include subject matter of the news.

11. A method as in claim 3, wherein said content comprises video or audio communication.

12. A method as in claim 6, wherein said measured parameters include duration of the communication, frequency of the communication and parties to the communication.

13. A method as in claim 1, wherein said mental state includes mental agility.

14. A method as in claim 1, wherein said mental state includes emotional state.
